(19) United States
(12) Patent Application Publication
(10) Pub. No.: US 2005/0222599 A1
(43) Pub. Date: Oct. 6, 2005

(54) Device for puncturing patient's skin

(75) Inventors: Andrzej Czernecki, Warsaw (PL); Wojciech Wyszogrodzki, Warsaw (PL); Wojciech Sarna, Warsaw (PL)

Correspondence Address: PILLSBURY WINTHROP SHAW PITTMAN LLP, 1650 TYSONS BOULEVARD, MCLEAN, VA, 22102, US

(73) Assignee: HTL-STREFA Spolka z o.o., PL 95-035 Ozorkow (PL)

(21) Appl. No.: 10/506,664

(22) PCT Filed: Mar. 5, 2003
(86) PCT No.: PCT/PL03/00019

§ 371 Date: May 2, 2005

(30) Foreign Application Priority Data
Mar. 6, 2002 (PL) P.352649
Mar. 7, 2002 (PL) P.352660
Mar. 22, 2002 (PL) P.352962
Aug. 28, 2002 (PL) P.355740

(51) Int. Cl. A61B 17/14; A61B 17/32; A61B 10/00

(52) U.S. Cl. 606/182; 600/567; 600/562; 600/564; 606/167; 604/185

(57) ABSTRACT

The device for puncturing comprises a sleeve (1), a push element (2) mounted on one end of the sleeve (1), a piston (5) with a puncturing tip (8) slidably mounted inside the sleeve (1), and a drive spring (10) positioned between a face (9) of the push element (2) and the piston (5), wherein the push element (2) comprises turnably mounted thereto a setting element (41) of the patient's skin piercing force having inwardly directed stair shaped members (42, 43) pressing the push rod (6) of the piston (5) in operation.

### BACKGROUND OF THE INVENTION

1. Field of the Invention

This invention relates to the device for puncturing patient's skin, in particular for blood sampling for the sake of diagnostic tests.

2. Description of the Related Art

The U.S. Pat. No. 5,356,420 discloses a puncturing device comprising a sleeve and a push element positioned at the first end of the sleeve. The other end of the sleeve ends with a bottom with an opening therein. Inside the sleeve a piston is slidably mounted, terminating with a push rod at the end closer to the push button, and with a puncturing tip at the end closer to the bottom opening. Inside the sleeve between the push element face and the piston, a drive spring is located, and between the piston and the sleeve bottom a return spring is placed. The piston comprises wings located on its outer perimeter which rest on an internal projection of the sleeve.

The European patent application No. 0885590 discloses a device for drawing blood samples with an adjustable attachment. The attachment is mounted onto the puncturing tip, and comprises a plurality of obliquely positioned grooves which are adapted to be joined with limiting elements on the sleeve external surface. Depending on the required depth of skin puncture by the lancet, the attachment is turned around its axis to a determined mark relating to the puncturing depth; then a finger is applied to the attachment opening, and the puncturing tip is released. The piercing depth depends on the settable distance between the lancet and the attachment end.

Further, the U.S. Pat. No. 5,613,978 discloses an adjustable tip for a puncturing device. In this solution setting of the puncturing depth by turning the tip around its axis is similar to that of the European patent application No. 0885590. The puncturing tip in the course of puncturing hits with its face surface against the flat face surface of the distal end of a cylindrical sleeve comprising an opening for the lancet in its wall. Moreover the adjustable tip has an intermediate cylindrical ring with adjusting screw members.

Further, the European patent application No. 1142534 discloses an assembly for adjusting the puncturing depth of a device comprising a sleeve, a piston with a puncturing tip slidably mounted in the sleeve, and a drive spring located between the push element face and the piston, wherein on the other sleeve end an adjustment ring is mounted with an opening for the puncturing tip, the adjustment ring having inwardly directed two half-ring members, oblique or stair shaped limiting members, which are hit by the piston fin in operation. Moreover, on the side surface of the adjustment ring a mark is located, while on the sleeve outer surface the scale of the puncturing depth is placed.

### SUMMARY OF THE INVENTION

According to the present invention, the device for puncturing patient's skin comprises a sleeve, a push element mounted on one end of the sleeve, a piston with puncturing tip slidably mounted inside the sleeve, and a drive spring positioned between the face of the push element and the piston, where at the other end of the sleeve an indicating-adjusting member comprising an opening for the puncturing tip is mounted turnably with respect to the axis of the sleeve, wherein the indicating-adjusting member has inwardly directed stair shaped limiting members, which are hit in operation by the fin of the piston, and has at least one indicator of the pre-set puncturing depth located in a circumferential groove in the lower part of the sleeve with external cut-outs. Preferably the external cut-outs have different widths.

According to yet another variety of the present invention, the push element comprises a turnably mounted therein puncturing force adjusting member, which comprises inwardly directed pair of oblique half-ring members pressing the push rod of the piston in operation.

According to yet another variety of the present invention, the push element comprises a turnably mounted therein puncturing force adjusting member, which comprises inwardly directed stair shaped members pressing the push rod of the piston in operation.

An advantage of the solution according to the present invention is a fact that the invention enables pre-setting of depth and/or force of puncturing of patient's skin for sampling patient's blood for the sake of diagnostic tests.

### BRIEF DESCRIPTION OF THE DRAWINGS

The accompanying drawings, which are incorporated in, and form a part of the specification, illustrate embodiments of the present invention and, together with the description, serve to explain the principles of the invention. In the drawings:

FIG. 1 shows longitudinal section of the first embodiment of the puncturing device according to the invention;

FIG. 2 shows view of the indicating-limiting member of the device of the FIG. 1 with limiting elements and indicators;

FIG. 3 shows general view of the device of the FIG. 1 with cut-outs for the indicator of the puncturing depth;

FIG. 4 shows longitudinal section of the second embodiment of the puncturing device according to the invention;

FIG. 5 shows view of the adjusting member for adjustment of the puncturing force with a pair of oblique half-ring members for the device from the FIG. 4;

FIG. 6 shows longitudinal section of the third embodiment of the puncturing device according to the invention; and

FIG. 7 shows view of the adjusting member for adjustment of the puncturing force with stepped elements for the device of the FIG. 6;
whereas the same elements of the puncturing devices depicted on the drawing have the same designations.

### DETAILED DESCRIPTION OF THE INVENTION

Reference will now be made in detail to the preferred embodiments of the invention, examples of which are illustrated in the accompanying drawings.

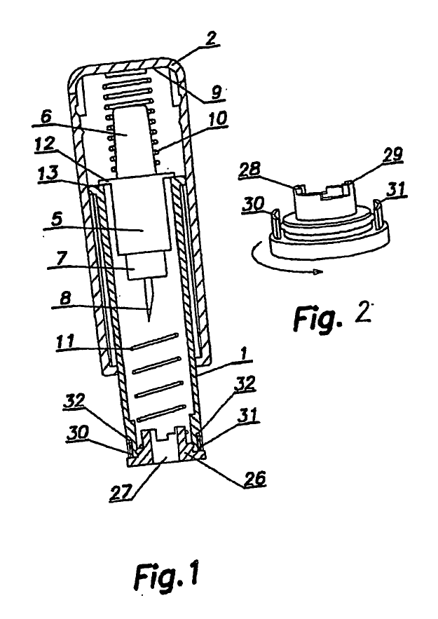

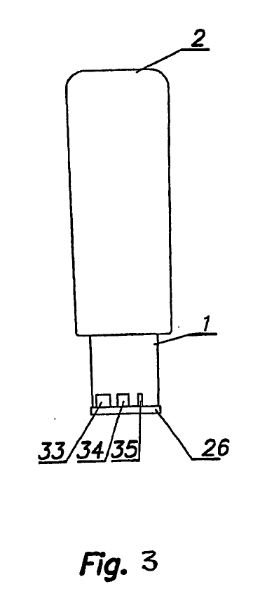

The device for puncturing the patient's skin in the first embodiment shown in the FIGS. 1 and 2 comprises the sleeve (1) and the push element (2) which is mounted on one of the sleeve (1) ends, and surrounds it on a substantial length. On the other end on the sleeve (1) an annular indicating-adjusting element (26) with an opening (27) therein is mounted turnably with respect to the axis of the sleeve (1). Inside the sleeve (1) the piston (5) is slidably inserted, which at the end proximal to the push element (2) ends with the push rod (6), and at the end proximal to the annular indicating-adjusting element (26) ends with the fin (7) with the puncturing tip (8). Inside the device between the face (9) of the push element (2) and the piston (5) the drive spring (10) is disposed, while inside the sleeve (1) between the piston (5) and the annular indicating-adjusting element (26) the return spring (11) is positioned. The piston (5) has in its upper portion outwardly directed wings (12) which rest on the upper edge (13) of the sleeve (1). The annular indicating-adjusting element (26) with the opening (27) therein for the puncturing tip (8) has stair shaped limiting members (28, 29) directed into the interior of the sleeve (1) which enable stepping adjustment of the puncture depth. Moreover the indicating-adjusting element (26) has indicators (30, 31) of the set puncture depth, which are positioned in a circumferential groove (32) in the lower portion of the sleeve (1) with external cut-outs (33, 34, 35), and are visible through the said cut-outs. Preferably the cut-outs (33, 34, 35) in the sleeve (1) have different widths, as shown in the FIG. 3.

Operation of the device according to this embodiment of the invention is as follows. The positioning of the device elements before use is shown in FIG. 1. The wings (12) of the piston (5) rest on the upper edge (13) of the sleeve (1) due to the pressure of the drive spring (10). Thus the piston (5) with the puncturing tip (8) is held in the first stable position. Pressing the push element (2) causes compression of the drive spring (10) until the face (9) of the push element (2) abuts against the push rod (6) of the piston (5). Further pressing of the push element (2) results in breaking off the wings (12) of the piston (5), and the drive spring (10) driving the piston (5) causes the fin (7) of the piston (5) to hit the internal member (14) limiting the puncturing depth, and the puncturing tip (8) passes through the opening (4) of the cap (3), and punctures the patient's skin to the depth pre-determined by the thickness of the cap (3).

Next the return spring (11) withdraws the piston (5) with the puncturing tip (8), which assumes the second stable position inside the sleeve (1). When the wings (12) of the piston (5) are broken off subsequent re-use of the device is not possible.

The depth of penetration of the patient's skin is set by turning the indicating-adjusting element (26) around the device axis, preferably in steps using a ratchet mechanism. This way the setting of the stair shaped limiting members (28, 29) is changed with respect to the fin (7) of the piston (5), and the depth of penetration of the patient's body by the puncturing tip (8) when the fin (7) of the piston (5) hits the said stepped limiting members. Moreover simultaneously the position of the penetration depth indicator (30, 31) changes, what is visible in one of the external cut-outs (33, 34, 35) in the sleeve (1), showing the depth of the puncture.

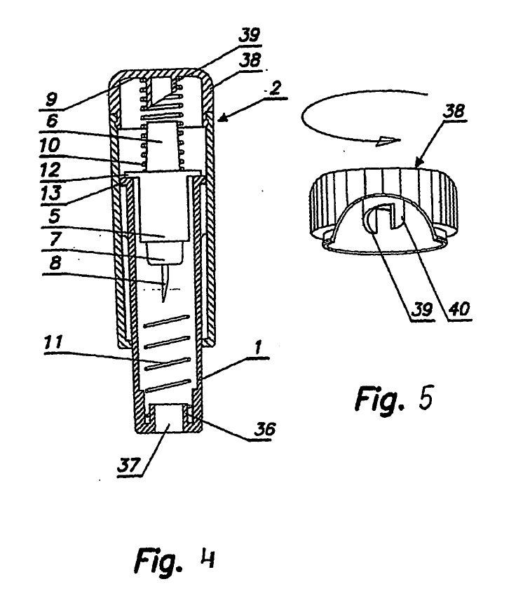

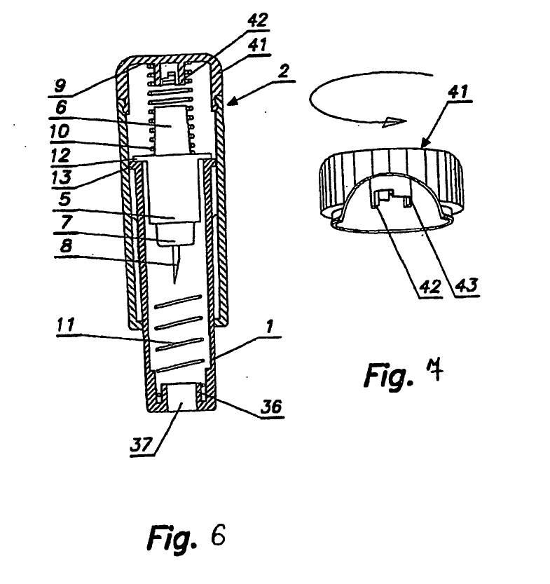

The device for puncturing the patient's skin in the second embodiment shown in the FIG. 4 comprises the sleeve (1) and the push element (2) which is mounted on one of the sleeve ends, and surrounds it on a substantial length. On the other end the sleeve ends with a limiting member (36) with an opening (37). Inside the sleeve (1) the piston (5) is slidably inserted, which at the end proximal to the push element (2) ends with the push rod (6), and at the end proximal to the limiting member (36) ends with the fin (7) with the puncturing tip (8). Inside the device between the face (9) of the push element (2) and the piston (5) the drive spring (10) is disposed, while inside the sleeve (1) between the piston (5) and the other end of the sleeve (1) the return spring (11) is positioned. The piston (5) has in its upper portion outwardly directed wings (12) which rest on the upper edge (13) of the sleeve (1). Moreover the push element (2) has a puncturing force setting element (38) which has inwardly directed pair of oblique half-ring members (39, 40) pressing the push rod (6) of the piston (5) in operation. The setting element (38) with the pair of oblique half-ring members (39, 40) is shown in a general view in the FIG. 5.

Operation of the device according to this embodiment of the invention is following. The positioning of the device elements before use is shown in FIG. 4. The wings (12) of the piston (5) rest on the upper edge (13) of the sleeve (1) due to the pressure of the drive spring (10). Thus the piston (5) with the puncturing tip (8) is held in the first stable position. Pressing the push element (2) causes compression of the drive spring (10) until the pair of the oblique half-ring members (39, 40) of the setting element (38) of the push element (2) abuts against the push rod (6) of the piston (5). Further pressing of the push element (2) results in breaking off the wings (12) of the piston (5), and the drive spring (10) driving the piston (5) causes the fin (7) of the piston (5) to hit the member (36) limiting the puncturing depth, and the puncturing tip (8) passes through the opening (37) of the limiting member (36), and punctures the patient's skin. Next the return spring (11) withdraws the piston (5) with the puncturing tip (8), which assumes the second stable position inside the sleeve (1).

Adjustment of the patient's skin puncturing force is accomplished by turning the setting element (38) around the device axis, preferably in steps using a ratchet mechanism. This way the distance of the oblique half-ring members (39, 40) from the push rod (6) of the piston (5) is changed, and hence the initial biasing force of the drive spring (10) at the moment of breaking the wings (12) off the piston (5), and hence the value of the force with which the puncturing tip (8) will pierce the patient's body.

The device for puncturing the patient's skin in the third embodiment is shown in the FIG. 6. This device is essentially built of the same elements as the device of the FIG. 4, but the push element (2) has differently shaped turnably mounted therein piercing force setting element (41) which has inwardly directed stair-shaped members (42, 43) pressing the push rod (6) of the piston (5) in operation. This setting element (41) with stair-shaped members (42, 43) is illustrated in a general view in the FIG. 7.

The operation of the device according to this embodiment is same as that of the device shown in the FIG. 4, and the setting of the patient's skin piercing force is also accomplished by turning the setting element (41) around the device axis. In this embodiment turning of the setting element (41) changes the distance of the pair of the stair-shaped members (42, 43) from the push rod (6) of the piston (5), and hence the initial biasing force of the drive spring (10) at the moment of breaking the wings (12) off the piston (5), and hence the value of the force with which the puncturing tip (8) will pierce the patient's body.

1. A device for puncturing patient's skin comprising a sleeve, a push element mounted on one end of the sleeve, a piston with puncturing tip slidably mounted inside the sleeve, and a drive spring positioned between the face of the push element and the piston, characterised by that at the other end of the sleeve (1) an indicating-adjusting member (26), comprising an opening (27) for the puncturing tip (8) and comprising inwardly directed stair shaped limiting members (28, 29), which are hit in operation by the fin (7) of the piston (5), is mounted turnably with respect to the axis of the sleeve (1), wherein the indicating-adjusting member (26) has at least one indicator (30, 31) of the pre-set puncturing depth located in a circumferential groove (32) in the lower part of the sleeve (1) with external cut-outs (33, 34, 35).

2. A device according to the claim 1 wherein the external cut-outs (33, 34, 35) in the sleeve (1) have different widths.

3. A device for puncturing patient's skin comprising a sleeve, a push element mounted on one end of the sleeve, a piston with puncturing tip slidably mounted inside the sleeve, and a drive spring positioned between the face of the push element and the piston, characterised by that the push element (2) comprises a turnably mounted therein puncturing force adjusting member (38), which comprises inwardly directed pair of oblique half-ring members (39, 40) pressing the push rod (6) of the piston (5) in operation.

4. A device for puncturing patient's skin comprising a sleeve, a push element mounted on one end of the sleeve, a piston with puncturing tip slidably mounted inside the sleeve, and a drive spring positioned between the face of the push element and the piston, characterised by that the push element (2) comprises a turnably mounted therein puncturing force adjusting member (38), which comprises inwardly directed stair shaped members (42, 43) pressing the push rod (6) of the piston (5) in operation.
